(19) United States
(12) Patent Application Publication
(10) Pub. No.: US 2007/0270818 A1
(43) Pub. Date: Nov. 22, 2007

(54) Connector apparatus

(75) Inventor: Alan Rezach, Atoka, TN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(73) Assignee: SDGI Holdings, Inc.

(21) Appl. No.: 11/409,823

(22) Filed: Apr. 24, 2006

(51) Int. Cl. A61F 2/30

(52) U.S. Cl. 606/61

(57) ABSTRACT

A connector apparatus for linking elongated members such as spinal rods includes, in one embodiment, a first member with a base and a post and a second member with a hole that fits over the post of the first member. The elongated members are situated generally between the members and astride the post. A locking member fits on the post and locks together the connector members and the elongated members.

The present disclosure broadly concerns spinal fixation systems and generally relates to a connector apparatus used to connect spinal rods. The apparatus can be useful for correction of spinal injuries or deformities.

### BACKGROUND

Several techniques and systems have been developed for use in correcting and stabilizing spinal curvatures, and for facilitating spinal fusion in the case of spinal disorders or degenerative conditions. In some systems, a pair of bendable rods may be longitudinally disposed adjacent the vertebral column and are fixed to various vertebrae along the length of the spine by way of a number of fixation elements, such as hooks and screws. In certain situations, it is desirable to supplement an existing spinal rod connected to the vertebral column with a new spinal rod, to add strength and stability to the fixation system.

Numerous spinal rod systems have been developed which provide transverse connectors for linking the adjacent spinal rods across the spinal midline to provide a rigid and stable construct. Such systems can present one or more difficulties for spinal surgeons. Many of the devices are high profile which increases soft tissue trauma and surgical complications. Moreover, it certain situations it is desirable to provide a transverse connection between adjacent spinal rods on the same side of the spinal midline.

Rigid transverse connections between spinal rods are beneficial because they restrict rod migration and increase construct stiffness. In many cases involving multi-level fusion of the spine, these features are essential while solid bone fusion is accomplished. In the post-operative period before fusion occurs, a significant amount of motion can occur between rods or other elongated members and other structure such as wires and hooks. That motion can, for example, allow a scoliotic correction to decrease or the pelvis to de-rotate towards a previous, deformed position. By providing a rigid transverse connection between two spinal rods, the loss of correction can be reduced and a stiffer construct can be created which may enhance the promotion of a solid fusion. A need remains for low profile devices which link adjacent spinal rods in an easy-loading, top-tightening fashion with a minimum of components and steps, providing increased stability to the fixation system.

### BRIEF DESCRIPTION OF THE DRAWINGS

FIG. 1 is a side elevational view of an embodiment of a connector device connected to elongated members.

FIG. 2 is a top plan view of the embodiment shown in FIG. 1.

FIG. 3 is a cross-sectional view of the embodiment of FIG. 1, taken along the lines 3-3 in FIG. 2 and viewed in the direction of the arrows.

FIG. 4 is a top plan view of an embodiment of a first member of the embodiment shown in FIG. 1.

FIG. 5 is a side elevational view of the embodiment shown in FIG. 4.

FIG. 6 is a side elevational view of an embodiment of a second member of the embodiment shown in FIG. 1.

FIG. 7 is a bottom plan view of the embodiment shown in FIG. 6.

FIG. 8 is a side elevational view of an embodiment of a locking member that may be used with the embodiments of a connector device.

FIG. 9 is a bottom plan view of another embodiment of a second member of an embodiment of a connector device.

FIG. 10 is a top plan view of another embodiment of a first member of an embodiment of a connector device.

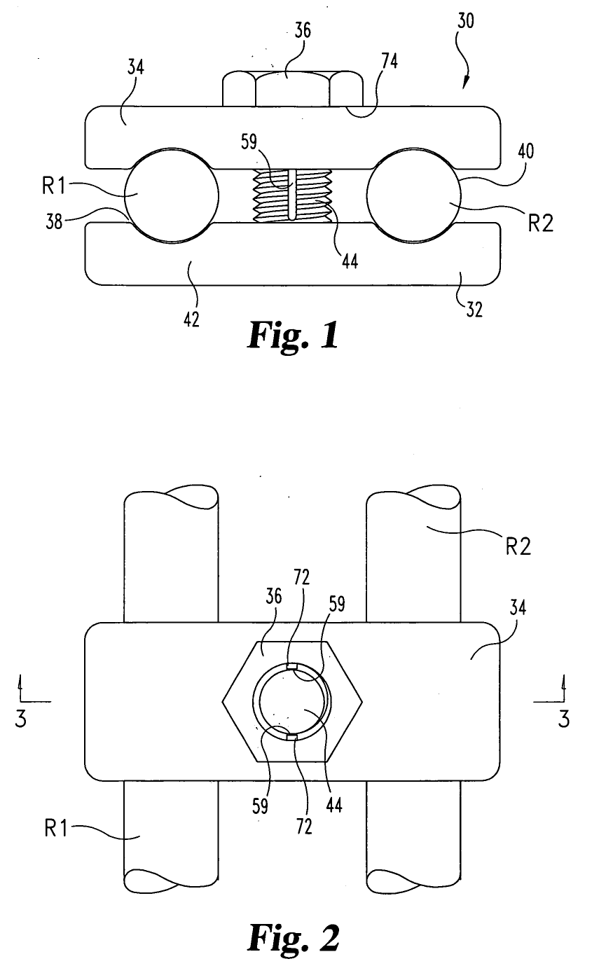

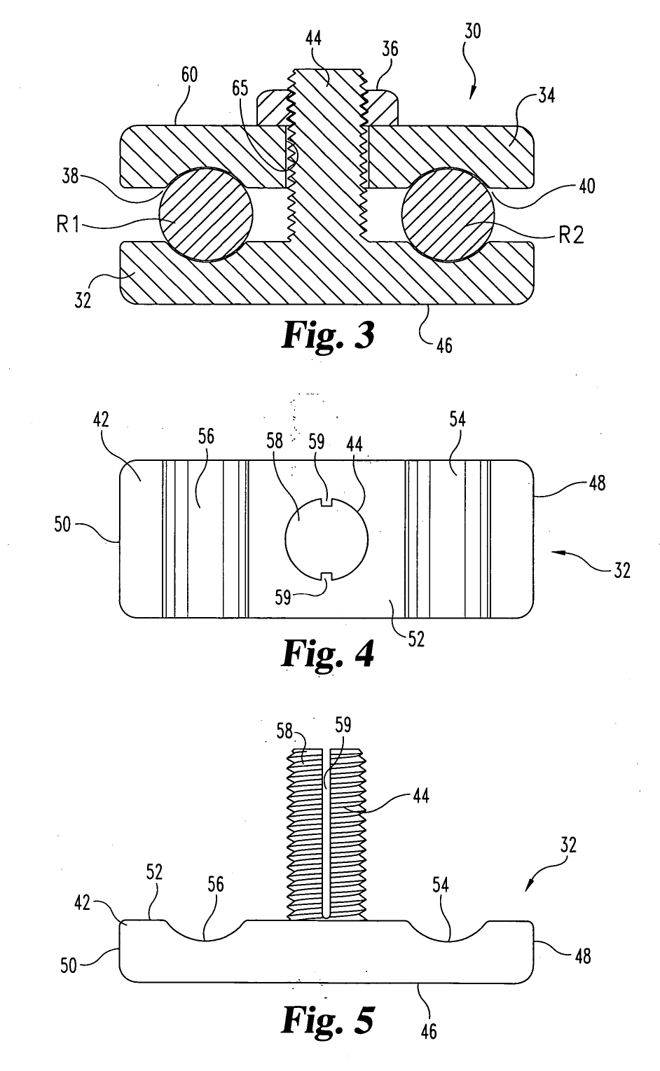

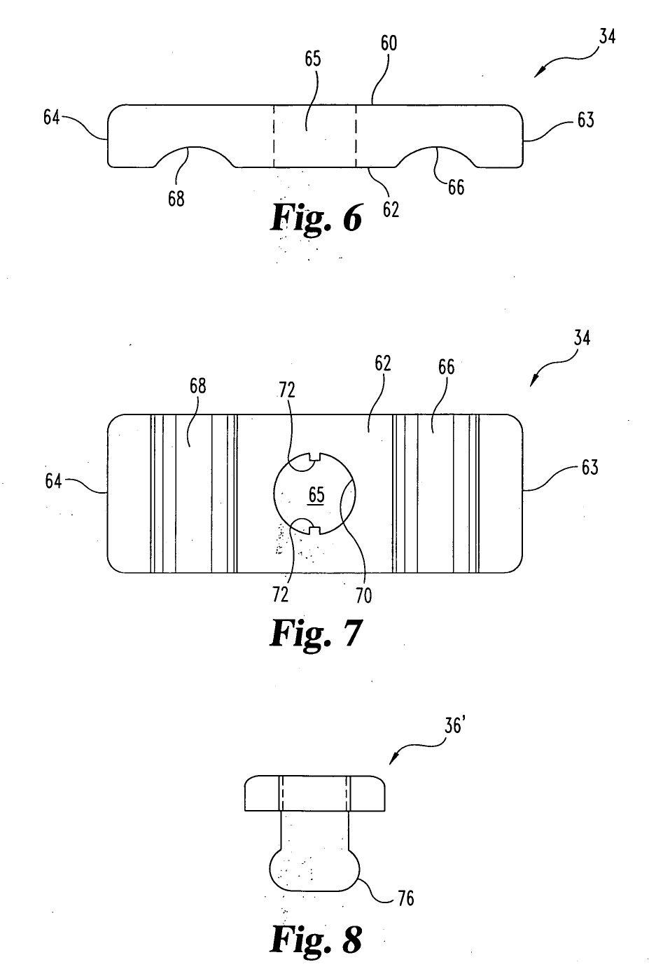

### DESCRIPTION OF THE ILLUSTRATED EMBODIMENTS

For the purpose of promoting an understanding of the principles of the disclosure, reference will now be made to the embodiments illustrated in the drawings and specific language will be used to describe the same. It will nevertheless be understood that no limitation of the scope of the claims is thereby intended, such alterations and further modifications in the illustrated device, and such further applications of the principles of the disclosure as illustrated therein, being contemplated as would normally occur to one skilled in the art to which the disclosure relates.

Referring generally to the figures, there is shown an embodiment of a connector apparatus 30. In that embodiment, apparatus 30 includes a first holding member 32, a second holding member 34, and a locking member 36. Generally, members 32 and 34 are assembled together to form passages 38 and 40 for rods R1 and R2, as further discussed below. Members 32 and 34 are secured together via locking member 36 in this embodiment.

Member 32 is generally T-shaped in the illustrated embodiment, having a base portion 42 and a post 44 extending from base 42. Post 44 may be formed integrally with each other, or in other embodiments may be separately fashioned and attached together, as by welding or other suitable attachment method. Post 44 is approximately centered with respect to portion 42 in the illustrated embodiment. In other embodiments, post 44 may be otherwise positioned, as for example placed toward one side of portion 42.

Base 42 is somewhat plate-like, having an external surface 46 and sides 48 and 50, and internal surface 52. At least part of surface 52 is concave in the illustrated embodiment, and in a particular embodiment surface 52 includes grooves or channels 54 and 56. As shown, channels 54 and 56 can be substantially cylindrical and have a diameter of a size sufficient to accommodate rods R1, R2 or other elongated members of various sizes. If a rod has a diameter smaller than or equal to the diameter of a channel (e.g. channel 54), then the rod fits within channel 54 and/or abuts a substantial portion of the surface of channel 54. If a rod has a diameter larger than the diameter of a channel, then the rod may contact the edges of the channel. Channels 54 and 56 are substantially parallel to each other in this embodiment, and are on either side of post 44. Channels 54 and 56 may be sized and configured essentially the same as each other, as shown in the illustrated embodiment, or one channel may be different from the other in terms of diameter, depth, or other dimension. Further, channels 54 and 56 could both be placed on one side of post 44, and/or may be non-parallel.

Post 44, as noted above, extends from base 42, and in the illustrated embodiment has an axis that is substantially perpendicular to portion 42. Post 44 may be threaded with standard machine threads substantially along its existing length from its free end 58 to base 42. Post 44 has a diameter of a size that will allow placement of a rod in each of channels 54 and 56. Thus, in the illustrated embodiment, post 44 does not impinge on either of channels 54 or 56. In other embodiments, however, post 44 could enter one or both of channels 54 or 56. In such cases, a rod may contact post 44 as well as the surface and/or edge(s) of its respective channel. The illustrated embodiment of post 44 includes two grooves 59 that extend from free end 58 toward base 42, and in a particular embodiment extends substantially all the way along post 44. In other embodiments, post 44 may have a single groove 59, more than two grooves 59, or may lack any such groove. Where multiple grooves 59 are provided in post 44, they may be irregularly spaced around post 44.

Second holding member 34 may be thought of as a cover or cap that fits around post 44. The illustrated embodiment of member 34 is a plate-like structure having an exterior surface 60, and interior surface 62, sides 63 and 64, and a hole 65. Exterior surface 60 is substantially planar in the illustrated embodiment, but may have a curve in other embodiments, and may be substantially smooth or roughened. For example, exterior surface 60 could be knurled for a high-friction connection with locking member 36. Interior surface 62 includes two channels 66 and 68, which can be substantially cylindrical and have a diameter of a size sufficient to accommodate rods or other elongated members of various sizes. Channels 66 and 68 are substantially parallel to each other and are on either side of hole 65. Channels 66 and 68 may be sized and configured essentially the same as each other, as shown in the illustrated embodiment, or one channel may be different from the other in terms of diameter, depth, or other dimension. Channels 66 and 68 could both be placed on one side of hole 65, and/or may be non-parallel. Further, one or both of channels 66 and 68 may be substantially identical to a respective facing channel 54 or 56. In the illustrated embodiment, for example, channel 66 is substantially identical to facing channel 54 at least in terms of diameter and depth, and channel 68 is substantially identical to facing channel 56 at least in terms of diameter and depth.

Hole 64 of member 34 may be substantially centered in member 34. In the illustrated embodiment, member 34 has a substantially smooth inner surface 70, and a pair of bosses 72 extend radially into hole 65. Bosses 72 are positioned in hole 65 so that they act with grooves 59 as a keying mechanism, ensuring that member 34 can be placed around post 44 only in particular orientation(s). In a particular embodiment, bosses 72 and grooves 59 are positioned with regard to their respective members 34 and 32 so that member 32 can be assembled with member 34 only when members 32 and 34 are substantially aligned with each other. In embodiments in which members 32 and 34 include one or more channels, bosses 72 and grooves 59 may be positioned so that such channels in each member are aligned with each other. In embodiments in which post 44 includes a different number of grooves 59, a number of bosses 72 equal to or less than the number of grooves 59 may be provided in hole 65, or hole 65 and post 44 could be otherwise keyed together. In embodiments in which post 44 does not have one or more grooves 59, hole 65 need not have one or more bosses 72.

Lock member 36 is an internally-threaded nut in the illustrated embodiment, with its internal threads being compatible with the machine threads on post 44. Lock member 36 may have an external hexagonal print for turning and tightening onto post 44. An underside 74 of lock member 36 may substantially conform to an adjacent portion of exterior surface 60 of member 34. Thus, where exterior surface 60 is substantially planar, underside 74 may also be substantially planar, and where exterior surface 60 has a curve adjacent hole 64 (e.g. a concave or conical surface), underside 74 may also have a substantially similar curve. Further, underside 74 may be smooth or roughened to provide close contact and/or high-friction contact with exterior surface 60. It will be seen that a variety of nut configurations could be used for lock member 36, such as a lock nut, or other types of a locking or holding member could be used, such as a press-on clamp or a shape-memory clamp.

Device 30 is generally used to connect two orthopedic elongated members, such as spinal rods, to each other. Particular embodiments of the use of device 30 are described below with respect to spinal surgical procedures. It will be understood that other uses could be made of device 30 in other orthopedic procedures.

In general, a surgeon first obtains access to the vertebrae or motion segment(s) to be instrumented via open, minimally-invasive or other techniques. A first rod R1 is connected to one or more vertebrae via bone anchors (not shown), which may be screws, hooks, clamps, other anchoring devices or a combination of them, and a second rod R2 is similarly connected to one or more of the same vertebrae as rod R1 or to a different set of vertebrae. Such attachment of rods via bone anchors to vertebrae can occur during the same surgery in which device 30 is implanted, or one or both of rods R1 and R2 can have been implanted in a surgery prior to introduction of device 30. In the latter case, it may be said that device 30 is placed in a “revision surgery,” i.e. one that places new implant(s), removes or makes adjustments to old implant(s), makes adjustments or further corrections to the spinal column, or otherwise revises a prior surgical procedure.

Member 32 of device 30 is placed with respect to rods R1 and R2 so that rods R1 and R2 are adjacent to interior surface 52 and post 44 is between rods R1 and R2. In embodiments in which member 32 includes channels 54 and/or 56, one or both of rods R1 and R2 may be placed at least partially in or adjacent to respective such channels. In situations in which rod R1 is connected to vertebrae in a previous surgical procedure or is otherwise connected to vertebrae prior to placement of device 30, then member 32 may be maneuvered around rod R1 so that rod R1 is adjacent to member 32 as indicated above. For example, a side (e.g. side 50) of member 32 may be moved between a previously-fixed rod R1 and adjacent bone or other tissue. Rod R2, which may be a “revision” rod, is then placed with respect to member 32 as indicated above. Rod R2 may be attached to vertebrae prior to or after connection to device 30. In situations in which rods R1 and R2 are being implanted during the same surgical procedure, device 30 may be placed with respect to rods R1 and R2 prior to the rods' connection to vertebrae, after connection of one rod to vertebrae but before the connection of the other to vertebrae, or may be placed with respect to rods R1, R2 after their connection to vertebrae.

Member 34 is placed over member 32 so that post 44 of member 32 extends through hole 64 of member 34. In embodiments in which post 44 includes one or more grooves 59 and one or more bosses 72 extend into hole 65, boss(es) 72 are aligned with groove(s) 59 so that boss(es) 72 slide along groove(s) as member 34 slides down along post 44. Member 34 is slid along post 44 so that it is adjacent or abutting rods R1 and R2, e.g. when rods R1 and R2 are at least partially in or adjacent to channels 66 and 68. When member 34 and member 32 are so positioned with respect to each other and rods R1 and R2, lock member 36 can be engaged with post 44. In the embodiment in which lock member 36 is a nut and post 44 is threaded, lock member 36 is threaded onto post 44 and against member 34 in order to press members 32 and 34 against rods R1 and R2. If the height of post 44 is such that a portion of post 44 sticks out from lock member 36 when device 30 is locked to rods R1 and R2, then the portion of post 44 that sticks out can be cut or clipped off or otherwise removed.

As previously noted, with device 30 two rods R1 and R2 can be connected together to form either a sturdy dual-rod support for one set of vertebrae or vertebral motion segments, or to form a longer “rod” by linking together two rods, one of which is attached to a superior set of vertebrae and the other of which is attached to an inferior set of vertebrae. Device 30 is easy to position with respect to a rod previously implanted, since it can be positioned with respect to such a rod in available spaces, i.e. without moving bone anchors to which the rod is connected or removing or disturbing bone or other tissue that has grown around or into parts of the rod or anchors. If necessary, bone or other tissue can be placed between members 32 and 34, such as in channels 54, 56, 66, and/or 68, and members 32 and 34 can be locked around it and the rod members to which it is attached. The open nature of device 30 with the separation of members 32 and 34 and the easy access to the interior of device 30 allow use of device 30 in a range of anatomical or surgical situations.

It will be understood that variations to the above-described structures can be made. For example, embodiments of post 44 may be relatively smooth or include other features that permit connection of a locking mechanism. If post 44 is smooth, then a clamp mechanism (e.g. a spring-loaded clamp) couldlbe placed around post 44 and atop member 34 so that members 32 and 34 are held around and/or to elongated members such as rods R1 and R2. Apertures or indentations could be provided in post 44 into which a pin or bar can be inserted to hold a locking mechanism to post 44 and against member 34. Further, in other embodiments there may be no groove in post 44 or bosses 72 on member 34. In such embodiments, a different keying mechanism may be employed, or no keying mechanism may be present, with the result that member 34 is freely rotatable with respect to post 44.

Further, where post 44 is threaded, an internally-threaded lock member 36′, shown in one embodiment in FIG. 8, could be provided that includes an undersurface or extension 76. Lock member 36′ not only exerts force on member 34 so that members 32 and 34 grip the elongated members, but also undersurface 76 contacts one or both elongated members. In this way, an additional point or points of engagement between the elongated members and connector 20 are provided, and the friction and grip strength of the connector can be increased. Undersurface 76 may be a part of a compressible portion, for example a portion similar to a leaf spring or a portion of elastically compressible material such as a rubber or elastic polymer.

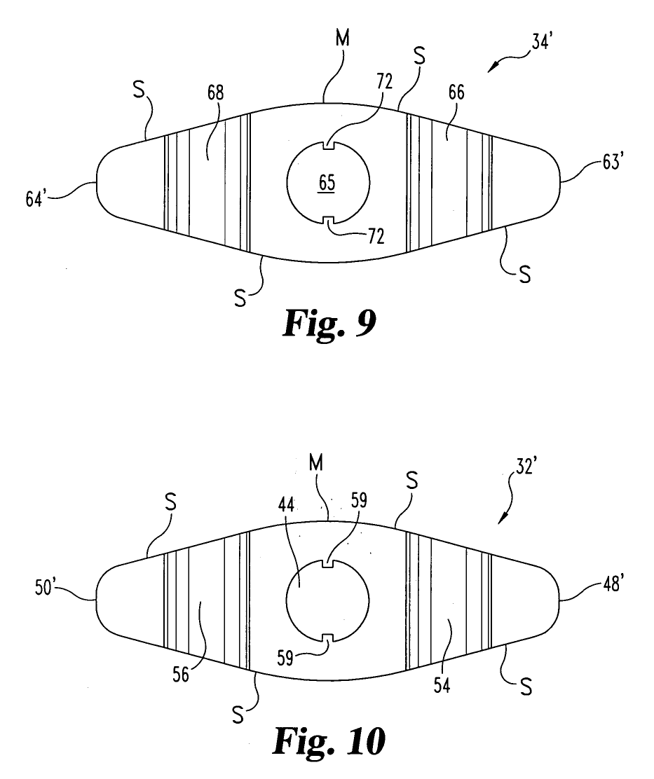

Other variations to the embodiments noted above are also possible. For example, members 32 and/or 34 may be substantially rectangular, or they may have other shapes. To make such members less bulky and less likely to interfere with or irritate adjacent tissue, members 32′ and/or 34′ may be substantially diamond-shaped, as seen in FIGS. 9-10. In that embodiment, members 32′ and 34′ are widest at a middle portion M proximate to hole 65 and post 44, and narrow to a relatively slim width at side portions 48′, 50′, 63′ and 64′ adjacent the elongated members. Such a diamond shape further provides surfaces S that are oblique to the longitudinal axis of an elongated member (shown in phantom). When flexion or extension of the vertebrae connected to the elongated member occurs, whether during surgery or in natural motion, stress on the elongated member is limited due to the non-perpendicular relationship between the elongated member and the adjacent surface S of member 32′ and 34′. It will be seen that members 32′ and 34′ may have substantially the same shape, or one may be somewhat differently shaped from the other. Other aspects of members 32′ and 34′ may be substantially the same as aspect described above with respect to members 32 and 34.

Additionally, as noted above, where channels (e.g. channels 54, 56, 66 and 68) are provided, they may be sized substantially identically to each other, or they may be sized somewhat differently. Thus, for example, channel 54 in member 32 and channel 66 in member 34 may be somewhat smaller in radius than channel 56 in member 32 and channel 68 in member 34. In that situation, a larger rod could be accommodated in the passage created by channels 56 and 68, and a smaller rod could be accommodated in the passage created by channels 54 and 66. Such a configuration will be useful in situations in which implantation of rods of two different diameters is indicated. Thus, for example, where a relatively thick rod is implanted in a first surgery, and relatively minimal revision is necessary, a somewhat thinner rod may be implanted in a revision surgery with an embodiment of connector 30 designed as previously noted. Similarly, where a relatively thin rod is first implanted, perhaps in a pediatric case, a later thicker rod may be implanted in a revision with such an embodiment of connector 30.

The parts of connector 30 are composed of biocompatible materials that are also compatible with particular elongated members or other implants with which connector 30 will be used. Thus, connector 30 may be made of titanium, nickel, alloys of titanium and nickel, stainless steel, certain sturdy plastic materials, or other sturdy materials. The material(s) chosen for connector device 30 should be the same as those of the rods with which connector device 30 is used, or at least of a material that will not cause discomfort or an adverse reaction when used with the rods. It will be appreciated that materials other than those described above could also be used.

While the disclosure has been illustrated and described in detail in the drawings and foregoing description, the same is to be considered as illustrative and not restrictive in character. It should be understood that only the preferred embodiments have been shown and described and that all changes and modifications that come within the spirit of the claims are desired to be protected.

What is claimed is:

1. A connector apparatus for linking elongated members, comprising:
a first member having a base portion and a post;
a second member having a hole, said members being connected so that said post extends through said hole and said members are movable with respect to each other, and said connected members defining a first location for accommodating a portion of a first elongated member and a second location for accommodating a portion of a second elongated member, said locations being between said members and on respective sides of said post; and
a locking member connected to said post and adapted to press said second member toward said first member.

2. The apparatus of claim 1, wherein at least one of said members includes at least one channel at at least one of said locations, said at least one channel being substantially linear and facing the other of said members, said at least one channel sized and configured to accommodate at least part of an elongated member.

3. The apparatus of claim 1, wherein said post is threaded, and said locking member is an internally-threaded nut.

4. The apparatus of claim 1, wherein said post includes at least one longitudinal groove, and said second member includes at least one boss extending into said hole and sized and configured to slide along said post groove.

5. The apparatus of claim 1, wherein said post includes two longitudinal grooves diametrically opposed to each other, and said second member includes two bosses extending into said hole and diametrically opposed to each other, said bosses being sized and configured to slide along respective ones of said post grooves.

6. The apparatus of claim 1, wherein said base portion of said first member has a central portion and two side portions, and said second member has a central portion and two side portions, and wherein at least one of said base portion of said first member and said second member have substantially a diamond shape in which the side portions are narrower than the central portion.

7. The apparatus of claim 1, wherein said base portion of said first member has a central portion and two side portions, said post being located in said central portion, and wherein a first of said side portions includes a first substantially linear channel at said first location that substantially faces said second member and is sized and configured to accommodate at least part of an elongated member, and a second of said side portions includes a second substantially linear channel at said second location that substantially faces said second member and is sized and configured to accommodate at least part of an elongated member.

8. The apparatus of claim 7, wherein said channels are substantially parallel.

9. The apparatus of claim 7, wherein said post is substantially between said channels.

10. The apparatus of claim 7, wherein second member has a central portion and two side portions, said hole being located in said central portion of said second member, and wherein said second member includes a third substantially linear channel at said first location that substantially faces said first channel and a fourth substantially linear channel at said second location that substantially said second channel, so that said first and third channels together form a passage for an elongated member, and said second and fourth channels together form a passage for an elongated member.

11. The apparatus of claim Error! Reference source not found., further comprising a first elongated member at least partially within one of said passages and a second elongated member at least partially within the other of said passages.

12. A spinal orthopedic implant apparatus, comprising:
first and second spinal rods;
a first connector member having a base portion and a central post, said base portion including first and second channels offset from said post, said first rods occupying at least a portion of said first channel and contacting said first connector member, said second rod occupying at least a portion of said second channel and contacting said first connector member, and said post extending substantially between said first and second rods;
a second connector member having a central hole and third and fourth channels offset from said hole, said third channel being approximately parallel to said first channel, and said fourth channel being approximately parallel to said second channel, and said post extending at least partially through said hole; and
a locking member fitted around at least a portion of said post and engaging said second connector member, whereby said first and second connector members and said first and second elongated members are substantially immovable with respect to each other.

13. The apparatus of claim 12, wherein said post and said second connector member include parts of a keying mechanism that substantially limits rotation of said second connector member with respect to said post, but does not limit translation of said second connector member with respect to said post.

14. The apparatus of claim 13, wherein said keying mechanism includes at least one substantially linear groove in said post and at least one boss on said second connector member positioned at least partially within said at least one groove.

15. The apparatus of claim 12, wherein said post is externally threaded and said locking mechanism is a nut.

16. The apparatus of claim 15, wherein said nut includes an undersurface that contacts at least one of said elongated members.

17. A method, comprising:
providing a connector having a first member with a base portion and a post, a second member having a hole therethrough, and a locking member;
bringing together said first member and a first spinal rod so that said first spinal rod is adjacent to or contacting said base portion;
placing a second spinal rod with respect to said first member so that said second spinal rod is adjacent to or contacting said base portion;
positioning said second member on said first member so that said post extends at least partially through said hole and so that said second member is adjacent to or contacting said first and second spinal rods; and
engaging said locking member to said post and said second member so that relative motion between said rods and said first and second members is substantially prevented.

18. The method of claim 17, wherein said first spinal rod is attached to the spine prior to said bringing together step, and said bringing together step includes moving said first member so that at least part of said base portion is between said first spinal rod and adjacent spinal tissue.

19. The method of claim 18, wherein said placing step occurs after said moving step.

20. The method of claim 17, wherein said providing, bringing together, placing and positioning steps occur prior to attachment of either of said rods to the spine.

21. The method of claim 20, further comprising:
connecting said locking member relatively loosely to said post after said providing, bringing together, placing and positioning steps; and
adjusting one or more of said first rod, said second rod, said first member and said second member relative to each other prior to said engaging step.

22. The method of claim 17, wherein following said bringing together step and said placing step, said post is between said rods.
